(19) United States
(12) Patent Application Publication
(10) Pub. No.: US 2010/0261734 A1
(43) Pub. Date: Oct. 14, 2010

(54) Methods of Treating Bladder Dysfunction Using Netupitant

(75) Inventors: Claudio Pietra, Como (IT); Sergio Cantoreggi, Vezia (CH); Riccardo Braglia, Lugano - Carabbia (CH)

Correspondence Address: PATENT CORRESPONDENCE;ARNALL GOLDEN GREGORY LLP, 171 17TH STREET NW, SUITE 2100, ATLANTA, GA, 30363, US

(73) Assignee: Helsinn Healthcare S.A., Pambio-Noranco (CH)

(21) Appl. No.: 12/758,479

(22) Filed: Apr. 12, 2010

Related U.S. Application Data
(60) Provisional application No. 61/169,127, filed on Apr. 14, 2009.

(51) Int. Cl. A61K 31/497; A61P 13/10

(52) U.S. Cl. 514/255.05

(57) ABSTRACT

The present invention relates to methods for reducing the frequency of bladder contractions without significantly affecting micturition pressure, comprising administering a pharmaceutical composition comprising a therapeutically effective amount of netupitant, or a pharmaceutically acceptable salt or prodrug thereof. The method can be used to treat bladder dysfunction without significantly increasing residual volume, including urgency, frequency, pollakiuria, nocturia, low deferment time, suboptimal volume threshold, neurogenic bladder, or combinations thereof.

### RELATED APPLICATIONS

This application claims priority to Provisional Application No. 61/169,127, filed Apr. 14, 2009. The contents of Provisional Application No. 61/169,127 are incorporated by reference in their entirety.

### FIELD OF THE INVENTION

The present invention relates to methods of treating bladder dysfunction with netupitant and its pharmaceutically acceptable salts and prodrugs.

### BACKGROUND OF THE INVENTION

The urinary bladder collects and stores urine until it is voided by micturition through the urethra. The bladder's ability to store and release urine is a result of the coordination of two muscles—the urethral sphincter muscle, which envelops the urethra and seals the urethra shut when the bladder is storing urine, and the detrusor muscle, which surrounds the bladder wall and squeezes urine out of the bladder during micturition. (Moore, Keith L.; Dalley, Arthur F, Clinically Oriented Anatomy, 5th Edition, 2006 Lippincott Williams & Wilkins).

In healthy adults, the storage and release of urine is mediated by a complex reflex system coordinated through the central nervous system. To store urine in the bladder, the brain signals the urethral sphincter to contract and seal the urethra shut. When the volume of urine stored in the bladder passes a certain threshold, receptors in the bladder send a signal to the central nervous system through afferent nerves that causes the detrusor muscle to contract and increase bladder pressure. This increase in bladder pressure causes another signal that indicates to the brain that there is a need to pass urine. In response to this signal, the nervous system produces an inhibitory signal that causes the urethral sphincter to remain contracted until such time as it is socially acceptable to urinate.

When it is appropriate to urinate, the brain sends another series of signals that enables the bladder to pass urine. One signal is sent to the sphincter, telling it to relax and open the urethra. A second signal is sent along efferent nerves to the detrusor, telling it to contract and squeeze the urine out through the urethra. (“Neurophysiology of Lower Urinary Tract Function and Dysfunction,” Naoki Yoshimura, MD, PhD and Michael B Chancellor, MD, Rev Urol. 2003; 5(Suppl 8): S3-S10).

Urologists recognize a variety of types of urinary dysfunction of varying physiological origin. For example, sudden and uncontrollable urges to urinate, commonly referred as urgency or urge incontinence, can be caused by detrusor muscle overactivity, which can cause the detrusor to contract at an inappropriately large amplitude or frequency in response to the afferent signal produced by normal bladder filling. The result of this contraction is an abnormal and sudden increase in bladder pressure, which can lead to involuntary discharge of urine or incontinence. Neurological imbalances also may exist between the strength of the signal that informs the brain of the need to urinate, and the strength of the inhibitory signal to the sphincter, producing an over-amplified need to urinate and preventing the sphincter from stopping involuntary discharges of urine. (“Neurogenic Bladder,” Raymond Rackley, MD, E-Medicine from WebMD, http://emedicine.medscape.com/article/453539-overview).

Overactive bladder is one of the most common types of bladder dysfunction, especially among the elderly, and it is characterized by symptoms of urgency, frequency, and incontinence, The drugs most prescribed for overactive bladder are the anticholinergics, and include oxybutinin, tolterodine and solifenacin. These drugs work mainly through the efferent cholinergic pathways to suppress muscle activity exerted by the detrusor muscle, thereby reducing the sense of urgency generated by the bladder feedback mechanism. A common side effect of these drugs, because of the weakened detrusor muscle, is an inability to completely void the bladder when urinating. This condition, known as urinary retention, severely limits the use of anti-cholinergics in the treatment of bladder dysfunction. (DITROPAN® (oxybutynin chloride) Prescribing Information, Ortho-McNeil Pharmaceutical, Inc., 2003; VESIcare® (solifenacin succinate) Prescribing Information, GlaxoSmithKline, 2005).

Other types of compounds have also been proposed for the treatment of urinary dysfunction, including hormone/estrogen therapies in women, 5-alpha reductase inhibitors, and neurokinin NK₁ receptor antagonists. At least one NK₁ receptor antagonist known as TAK-637, has been studied in an animal model for overactive bladder (Jpn. J. Pharmacol. 86, 165-169 (2001)), but to date TAK-637 remains unapproved, and the anticholinergics remain the principal pharmacological means for treating urinary dysfunction. As a result, there remains a need for drugs to treat one or more bladder dysfunctions, preferably without the significant side effects caused by anticholinergics and other current therapies.

### SUMMARY OF THE INVENTION

It has been discovered that netupitant, a novel NK₁ antagonist developed by Hoffmann-La Roche, Inc., interrupts the reflective bladder feedback mechanism induced by bladder distension, and selectively reduces the frequency of bladder contractions caused by such bladder distension without decreasing the amplitude of micturition pressure in the bladder, or significantly increasing the retention of urine in the bladder during micturition (i.e. urinary retention). Thus, a treatment is provided using netupitant that can ameliorate a number of the conditions associated with bladder dysfunction, such as urgency, frequency and incontinence, while preserving the muscular function in the bladder, and the ability of the bladder to completely void its contents.

Therefore, in one embodiment, a method is provided for interrupting premature reflexive bladder signaling induced by bladder distension, and thereby reducing the frequency of bladder contractions in a human patient in need of such reduction, without significantly affecting micturition pressure needed for bladder voiding, comprising administering to said patient a therapeutically effective amount of netupitant or a pharmaceutically acceptable salt or prodrug thereof, wherein said therapeutically effective amount is sufficient to significantly reduce the frequency of bladder contractions induced by bladder distention, without significantly reducing micturition pressure needed for bladder voiding.

In another embodiment, a method is provided for treating bladder dysfunction associated with urinary retention, in a human patient for whom anticholinergics are contraindicated due to the urinary retention, wherein said bladder dysfunction is characterized by urinary urgency and, optionally, urinary frequency and incontinence, comprising administering to a human patient in need of such treatment a therapeutically effective amount of netupitant, or a pharmaceutically acceptable salt or prodrug thereof, wherein said therapeutically effective amount is sufficient to significantly reduce episodes of said urgency during a 24-hr time period without significantly increasing said urinary retention when the bladder is voided voluntarily.

It will also be appreciated that the methods of the invention can be used in the treatment of numerous urinary disorders, and in yet another embodiment, a method is provided for treating a human patient in need of treatment for bladder dysfunction, wherein said bladder dysfunction is selected from urgency, frequency, pollakiuria, nocturia, low deferment time, suboptimal volume threshold, and neurogenic bladder, or a combination thereof, comprising: administering to said patient a therapeutically effective amount of netupitant, or a pharmaceutically acceptable salt or prodrug thereof, wherein said amount is preferably effective to treat said bladder dysfunction without significantly increasing residual volume.

### BRIEF DESCRIPTION OF THE DRAWINGS

FIG. 1 is a graph depicting the pharmacokinetic profile of netupitant in humans following oral administration of a single oral dose.

### DETAILED DESCRIPTION OF THE INVENTION

The present invention may be understood more readily by reference to the following definitions and detailed description of preferred embodiments of the invention and the non-limiting Examples included therein.

Improved methods have been developed for treating urinary bladder dysfunctions with netupitant and its pharmaceutically acceptable salts and prodrugs. It has now been discovered that netupitant can be used to effectively treat a recognized subset of patients that suffer from bladder dysfunction such as urgency, frequency, pollakiuria, nocturia, low deferment time, suboptimal volume threshold, and neurogenic bladder, and combinations thereof. The methods can be practiced without affecting the residual volume, and therefore can be practiced in patients with symptoms of urinary retention, for whom anticholinergics are contraindicated.

“Netupitant” is a selective NK₁ receptor antagonist of the formula 2-[3,5-bis(trifluoromethyl)phenyl]-N,2-dimethyl-N-[4-(2-methylphenyl)-6-(4-methylpiperazin-1-yl)pyridin-3-yl]propanamide, or Benzeneacetamide, N,α,α-trimethyl-N-[4-(2-methylphenyl)-6-(4-methyl-1-piperazinyl)-3-pyridinyl]-3,5-bis(trifluoromethyl)-, having a molecular weight of 478.61, CAS registry number 290297-26-6, and the below chemical structure:

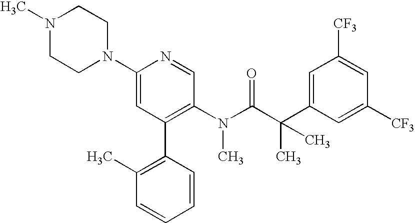

Both netupitant, its pharmaceutically acceptable salts, and its pharmaceutically acceptable prodrugs can be used in the methods of the present invention for the treatment of bladder dysfunctions.

As used herein, the term “pharmaceutically acceptable salt” refers to a salt of a compound to be administered prepared from pharmaceutically acceptable non-toxic acids. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like.

As used herein, the term “prodrug” refers to a chemical derivative of netupitant that degrades to netupitant in vivo via spontaneous or enzymatic transformation. Prodrugs are designed to overcome pharmaceutically and/or pharmacokinetically based problems associated with the parent drug molecule that might otherwise limit the clinical usefulness of the drug, especially due to drug solubility issues in the case of netupitant. Prodrugs of netupitant are described in U.S. Pat. No. 6,593,472, the contents of which are hereby incorporated by reference.

As used herein, “therapeutically effective amount” refers to an amount sufficient to elicit the desired biological response. The therapeutically effective amount or dose will depend on the age, sex and weight of the patient, and the current medical condition of the patient. The skilled artisan will be able to determine appropriate dosages depending on these and other factors in addition to the present disclosure.

When dose amounts are expressed herein in reference to a salt or prodrug of netupitant, it will be understood that the amount expressed is based on the corresponding amount of the free base of netupitant. Thus, for a prodrug with a molecular weight of 600, if this document referred to the administration of 100 mg of this netupitant or a prodrug thereof, it would be understood that 125.36 mg of the prodrug is administered, since the molecular weight of the prodrug is 125.36% of the molecular weight of the netupitant base.

The terms “treating” and “treatment,” when used herein, refer to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “significantly” preferably refers to a level of statistical significance. The level of statistical significant can be, for example, of at least p<0.05, of at least p<0.01, of at least p<0.005, or of at least p<0.001. When a measurable result or effect is expressed or identified herein, it will be understood that the result or effect can be evaluated based upon its statistical significance relative to a baseline.

The phrase “frequency of bladder contractions,” when used herein, refers to the frequency at which the detrusor muscle contracts, usually in response to bladder distension. Bladder contractions are believed to be the primary mechanism by which the bladder signals to the brain the need of micturition.

The phrase “micturition pressure,” when used herein, refers to the pressure generated by the detrusor when it contracts to expel urine through the urethra after the filling phase is completed.

The phrase “bladder dysfunction,” when used herein, refers to any disorder that results in the bladder's inability to properly store urine or release urine at a socially acceptable time, place, and/or frequency. Non-limiting examples of bladder dysfunctions include urgency, frequency, pollakiuria, nocturia, low deferment time, suboptimal volume threshold, neurogenic bladder, and bladder outflow obstruction.

The phrase “bladder distension,” when used herein, refers to the filling of the bladder with urine, and the stretching of the bladder in response to the filling.

The phrase “residual volume,” when used herein, refers to the volume of urine that remains in the bladder after voluntary urination, and is typically given as an average value based upon multiple urinations.

The phrase “volume threshold,” when used herein, refers to the volume of urine in the bladder that triggers a signal to the brain of the need to urinate.

The phrase “urinary retention,” also known as ischuria, is a lack of ability to urinate or to void the bladder volume completely during urination.

The phrase “deferment time,” when used herein, refers to the amount of time between the first desire to urinate and the voluntary release of urine from the bladder.

The phrase “urgency” or “urinary urgency,” when used herein, refers to the sudden, compelling need to urinate. Urgency can be measured by a variety of methods, including the number of micturitions over a period of time (usually 24 hours), the number of incontinent episodes over a period of time (usually between 24 hours and 1 week), and deferment time.

The phrase “frequency” or “urinary frequency,” when used herein, refers to the number of micturitions over a given period of time, typically 24 hours.

The phrase “bladder outflow obstruction,” when used herein, refers to the partial blockage of the urethra. Non-limiting examples of bladder outflow obstructions include benign prostatic hyperplasia (BPH), prostate cancer, and kidney stones.

The phrase “overactive bladder,” when used herein, refers to bladder dysfunction, regardless of the cause, that is characterized by urinary urgency, with or without urge incontinence, usually with frequency and nocturia.

The phrase “neurogenic bladder,” when used herein, refers to bladder dysfunction characterized by urinary incontinence caused by an imbalance between the strength of signal indicating the need to urinate and the strength of the signal inhibiting the relaxation of the urethral sphincter. Neurogenic bladder usually is associated with a neurological dysfunction such as multiple sclerosis or Parkinson's Disease.

The phrase “suboptimal” is used herein with reference to bladder volume thresholds, and when used herein refers to an amount that is significantly below a reference amount derived from a corresponding population of apparently healthy human subjects. In particular populations, a “suboptimal volume threshold” can be an amount less than 350 ml, an amount less than 300 ml, an amount less than 250 ml, an amount less than 200 ml, or an amount less than 150 ml. (“Terminology in Urodynamics as defined by The International Continence Society,” http://www.sghurol.demon.co.uk/urod/ics.htm).

The phrase “excessive” is used herein with reference to residual volume of urine in the bladder, and when used herein refers to an amount that is significantly above a reference amount derived from a corresponding population of apparently healthy human subjects. In particular populations, an “excessive residual volume” can be an amount greater than 25 ml, an amount greater than 50 ml, an amount greater than 75 ml, an amount greater than 100 ml, an amount greater than 125 ml, or an amount greater than 150 ml. (“Evaluation of Voiding Dysfunction and Measurement of Bladder Volume,” Christopher E Kelly, MD, Rev Urol. 2004; 6(Suppl 1): S32-S37).

Throughout the description and claims of this specification, the word “comprise” and variations of the word, such as “comprising” and “comprises,” means “including but not limited to,” and is not intended to exclude, for example, other additives, components, integers or steps.

As used in the specification and the appended claims, the singular forms “a,” “an” and “the” or like terms include plural referents unless the context clearly dictates otherwise. Thus, for example, reference to “a pharmaceutical carrier” includes mixtures of two or more such carriers, and the like. The word “or” or like terms as used herein means any one member of a particular list and also includes any combination of members of that list.

Pharmaceutical Compositions

The pharmaceutical compositions used in various embodiments of the invention include netupitant, or a pharmaceutically acceptable salt or prodrug thereof. The active ingredient will typically be incorporated into pharmaceutical preparations for ease of handling and administration. The pharmaceutical preparations can be administered orally, e.g. in the form of tablets, coated tablets, dragees, and soft gelatine capsules, solutions, emulsions or, suspensions.

Netupitant as well as its pharmaceutically acceptable salts and prodrugs can be processed with pharmaceutically inert, inorganic or organic excipients for the production of tablets, coated tablets, dragees and hard gelatine capsules. Lactose, corn starch or derivatives thereof, talc, stearic acid or its salts etc can be used as such excipients e.g. for tablets, dragees and hard gelatine capsules.

Suitable excipients for soft gelatine capsules are e.g. vegetable oils, waxes, fats, semi-solid and liquid polyols etc.

Suitable excipients for the manufacture of solutions and syrups are e.g. water, polyols, saccharose, invert sugar, glucose etc.

Suitable excipients for injection solutions are e.g. water, alcohols, polyols, glycerol, vegetable oils etc.

Moreover, the pharmaceutical preparations can contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances. The pharmaceutical compositions can be included in a container, pack, or dispenser together with instructions for administration.

The dosage can vary within wide limits and will, of course, be fitted to the individual requirements in each particular case. In general, in the case of oral administration a daily dosage of about 10 to 600 mg per person should be appropriate, although the above upper limit can also be exceeded when necessary. The dose can be administered in a single daily dosage, due to the excellent pharmacokinetics observed for the molecule.

In one embodiment, the dosage is in the range of about 0.05 mg to about 10 mg per kg of the human patient. In another embodiment, the dosage is in the range of about 0.1 mg to about 3 mg per kg of the human patient.

Methods of Treatment

As discussed above, it has now been discovered that a particular NK₁ receptor antagonist, netupitant, can be used to effectively treat a recognized subsets of patients that suffer from bladder dysfunction, including those that have symptoms of urinary retention. This discovery is based on the results of experimentation described in the “Examples” below, which revealed that netupitant, unlike some other NK₁ receptor antagonists, affects the mechanistic workings of the bladder in a unique way that can be very useful in treating specific types of urinary dysfunction.

Thus, in one embodiment, a method is provided for interrupting premature reflexive bladder signaling induced by bladder distension, and thereby reducing the frequency of bladder contractions in a human patient in need of such reduction, without significantly affecting micturition pressure needed for bladder voiding, comprising administering to said patient a therapeutically effective amount of netupitant or a pharmaceutically acceptable salt or prodrug thereof, wherein said therapeutically effective amount is sufficient to significantly reduce the frequency of bladder contractions induced by bladder distention, without significantly reducing micturition pressure needed for bladder voiding. The method can be used when said patient suffers from urinary urgency with symptoms of urinary retention, and optionally urinary frequency and nocturia. The method also can be used when said patient suffers from suboptimal volume threshold and/or excessive residual volume, wherein said amount is effective to significantly increase said volume threshold. The methods can reduce the frequency of bladder contractions by at least 20%, at least 30%, at least 40%, or at least 50% as compared to baseline. Furthermore, the methods can reduce micturition pressure by less than 5%, less than 10%, less than 15%, or less than 20% as compared to baseline.

These results also suggest that netupitant reduces symptoms of urgency without inhibiting voiding function, presumably due to its ability to act on the afferent pathway of the micturition reflex, rather than the efferent pathway, an ability that is not found in all NK₁ receptor antagonists. Thus, in another embodiment, a method is provided for treating bladder dysfunction associated with urinary retention in a human patient for whom anticholinergics are contraindicated, wherein said bladder dysfunction is characterized by urinary urgency and, optionally, urinary frequency and incontinence, comprising administering to a human patient in need of such treatment a therapeutically effective amount of netupitant, or a pharmaceutically acceptable salt or prodrug thereof, wherein said therapeutically effective amount is sufficient to significantly reduce episodes of said urgency during a 24-hr time period without significantly increasing said urinary retention when the bladder is voided voluntarily.

The method can be used when said bladder dysfunction is characterized by urinary urgency and/or urinary frequency. In particular embodiments, the amount is effective to reduce said urinary urgency and urinary frequency, effective to increase the volume threshold of the bladder and to reduce the incidence of premature contractions induced by bladder distension, effective to increase deferment time, or combinations thereof.

These methods can further include, before the step of administering the composition, diagnosing said subject as having overactive bladder, and diagnosing said subject as having urinary retention. In certain embodiments, the methods can further include, before the step of administering the composition, diagnosing said subject as having a bladder outflow obstruction such as benign prostatic hyperplasia (BPH). The methods can reduce average urinary frequency by at least 10%, at least 20%, at least 30%, or least 40% as compared to baseline. The methods can reduce average deferment time by at least 20%, at least 30%, at least 40%, or least 50% as compared to baseline. Furthermore, the methods can increase average residual volume by less than 5%, less than 10%, less than 15%, or less than 20% as compared to baseline. Finally, the methods can increase the average volume urinated by up to 10%, 25%, 40% or even 60% as compared to a baseline.

Because netupitant can reduce these symptoms, it is suggested that netupitant can treat a variety of specific bladder dysfunctions. Thus, in yet another embodiment, a method is provided for treating a human patient in need of treatment for bladder dysfunction, wherein said bladder dysfunction is selected from urgency, frequency, pollakiuria, nocturia, low deferment time, suboptimal volume threshold, and neurogenic bladder, or a combination thereof, comprising: administering to said patient a therapeutically effective amount of netupitant, or a pharmaceutically acceptable salt or prodrug thereof, wherein said amount is effective to treat said bladder dysfunction, preferably without a corresponding significant increase in residual volume or urinary retention.

### Example 1

Objective

The effect of netupitant (N) was studied on cystometric parameters in acetic acid (AA)-induced overactive bladder (OAB) in anesthetized guinea-pigs. Results were compared with those obtained with a control and L-733,060 (L), another potent and selective NK1 receptor antagonist.

Methods

Adult female guinea-pigs were anesthetized with urethane, and the jugular vein and urinary bladder were catheterized for drug administration and intravesical pressure recording, respectively. The urinary bladder was continuously infused with either saline or 0.2% AA. After 30 min stabilization, N, L or their vehicle (glucose 5%) were administered in separate animals (n=10 per group). Intercontraction interval (ICI, sec), and amplitude of micturition (AM, mmHg) were analyzed at 10, 20, 40 and 60 min post administration. For each dose, maximal effects were expressed as % of variation from respective basal values. ED50% (doses inducing a 50% increase in ICI) were calculated using linear regressions.

Results

In comparison to saline bladder infusion, 0.2% AA significantly (p<0.001) reduced ICI (−70%) and increased AM (+164%). In comparison, administration of vehicle was ineffective on cystometric parameters.

Starting from the dose of 0.1 mg/kg, N dose-dependently and significantly increased ICI starting from 10 min and up to 60 min post-administration. Maximal ICI increases were +51±15, +44±8, +69±8 and +96±20% at 0.1, 0.3, 1 and 3 mg/kg, respectively. The calculated ED50% for N was 0.62 mg/kg i.v.

N was devoid of significant effect on AM, the maximal effect was observed at 1 mg/kg, 20 min after administration (−13±6%).

L dose-dependently and significantly increased ICI starting from 10 min and up to 60 min after administration. Maximal effects at the doses of 3 and 10 mg/kg were +40±10 and +76±11%, respectively. The calculated ED50% of L was 5.13 mg/kg i.v. In addition, L at 10 mg/kg decreased significantly AM (p<0.05 versus basal values). Maximal effect reached −28±5%, 10 min after administration.

Conclusion

In guinea-pigs, 0.2% AA induced OAB characterized by a decreased ICI. A great increase of AM was also observed.

In animals treated with 0.2% AA, both NK1 receptor antagonists, N and L dose-dependently increased ICI, probably acting NK1 receptor antagonists mainly located on spinal afferent fibers. L but not N decreased AM, perhaps acting on efferent fibers. In view of ED50% values, N is about 16 fold more potent than L.

In conclusion, N can be useful for the treatment of OAB in humans. Since it was without effect on bladder contractility, it can be used without increasing of residual volume as observed with antimuscarinics in clinical practice.

### Example 2

Objective and Methods

The pharmacokinetic profile of netupitant (N) in humans was examined in a randomised, double-blind, placebo controlled study comprising 5 dose groups (10, 30, 100, 300 and 450 mg) with 6 subjects in each group, of whom four received netupitant and two received placebo.

Results

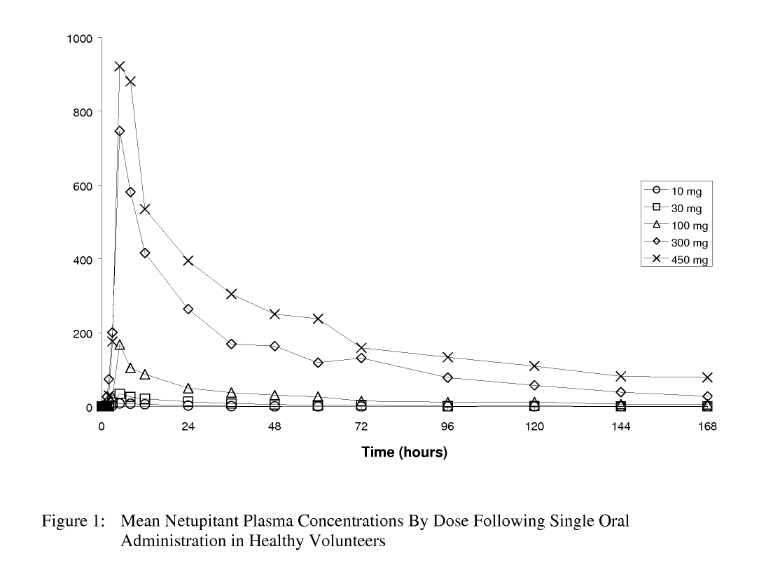

Following oral administration of netupitant, there was a lag of between 45 minutes and 3 hours before measurable amounts of the drug could be detected in plasma. After this lag time, the plasma concentration of netupitant increased in a first order fashion, with maximum levels reached at approximately 5 hours post-dose. The mean plasma concentration-time profiles for each dose are shown in FIG. 1.

Plasma exposure, judged by maximum concentration (Cmax) and AUC, increased super-proportionally with dose. Table 1 below summarizes the pharmacokinetic parameters associated with each dose.

TABLE 1
Summary of the Mean (CV %) Pharmacokinetics of Netupitant in Healthy
Volunteers Following Oral Administration (N = 4 per dose)

| Parameter | 10 mg | | 30 mg | | 100 mg | | 300 mg | | 450 mg | |
|---|---|---|---|---|---|---|---|---|---|---|
| Cₘₐₓ (ng/mL) | 8.76 | (32.4) | 36.0 | (15.9) | 168 | (22.1) | 747 | (26.9) | 1134 | (30.8) |
| AUC_{inf} (h · ng/mL) | 233 | (20.4) | 995 | (55.8) | 4800 | (27.3) | 25200 | (24.9) | 43700 | (19.5) |
| AUCₗₐₛₜ (h · ng/mL) | 140.2 | (61.2) | 870 | (58.7) | 4360 | (26.1) | 23100 | (23.0) | 36600 | (19.0) |
| t_{lag} (h) | 1.88 | (45.5) | 1.75 | (16.5) | 1.88 | (13.3) | 0.81 | (63.4) | 1.26 | (39.6) |
| tₘₐₓ (h) | 5.75 | (26.1) | 5.76 | (26.2) | 5.00 | (0.0) | 5.00 | (0.0) | 5.76 | (26.2) |
| t_{1/2} (h) | 16.0 | (6.9) | 29.1 | (45.8) | 54.2 | (33.1) | 48.1 | (32.2) | 59.7 | (31.2) |
| Kₑₗ (l/h) | 0.0435 | (6.9) | 0.0270 | (35.8) | 0.0139 | (33.4) | 0.0154 | (26.3) | 0.0124 | (27.1) |
| V_{z}/F (L) | 1010 | (12.9) | 1320 | (20.6) | 1710 | (40.3) | 850 | (31.8) | 881 | (18.8) |
| Cl/F (L/h) | 44.0 | (19.5) | 35.5 | (36.3) | 22.1 | (27.6) | 12.6 | (28.3) | 10.6 | (20.7) |

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All references cited herein, including patents, patent applications, and published patent applications, are hereby incorporated by reference in their entireties, whether or not each is further individually incorporated by reference.

What is claimed is:

1. A method of interrupting premature reflexive bladder signaling induced by bladder distension, and thereby reducing the frequency of bladder contractions in a human patient in need of such reduction, without significantly affecting micturition pressure needed for bladder voiding, comprising:
administering to said patient a therapeutically effective amount of netupitant or a pharmaceutically acceptable salt or prodrug thereof, wherein said therapeutically effective amount is sufficient to significantly reduce the frequency of bladder contractions induced by bladder distention, without significantly reducing micturition pressure needed for bladder voiding.

2. The method of claim 1, wherein said patient suffers from urinary urgency and said urinary urgency is associated with urinary retention, and optionally urinary frequency and nocturia.

3. The method of claim 1, wherein said patient suffers from suboptimal volume threshold and excessive residual volume, wherein said amount is effective to increase said volume threshold.

4. The method of claim 1, wherein said therapeutically effective amount reduces the frequency of bladder contractions by at least 40%.

5. The method of claim 1, wherein said therapeutically effective amount reduces the micturition pressure by less than 15%.

6. The method of claim 1, wherein said netupitant is administered orally, in the form of a tablet, capsule or liquid, and said therapeutically effective amount comprises from 0.05 mg to 10 mg of netupitant, or its pharmaceutically acceptable salt or prodrug, per kg of the human patient.

7. A method of treating bladder dysfunction associated with urinary retention in a human patient for whom anticholinergics are contraindicated, wherein said bladder dysfunction is characterized by urinary urgency and, optionally, urinary frequency and incontinence, comprising:
administering to a human patient in need of such treatment a therapeutically effective amount of netupitant, or a pharmaceutically acceptable salt or prodrug thereof,
wherein said therapeutically effective amount is sufficient to significantly reduce episodes of said urgency during a 24-hr time period without significantly increasing said urinary retention when the bladder is voided voluntarily.

8. The method of claim 7, wherein said bladder dysfunction is characterized by urinary urgency and urinary frequency, and said therapeutically effective amount is sufficient to reduce said urinary urgency and urinary frequency.

9. The method of claim 7, wherein said amount is effective to increase the volume threshold of the bladder, and to reduce the incidence of premature contractions induced by bladder distension.

10. The method of claim 7, wherein said amount is effective to increase deferment time.

11. The method of claim 7, further comprising, before the step of administering the netupitant or pharmaceutically acceptable salt or prodrug thereof,
diagnosing said subject as having overactive bladder, and
diagnosing said subject as having urinary retention.

12. The method of claim 7, further comprising, before the step of administering the composition,
diagnosing said subject as having a bladder outflow obstruction.

13. The method of claim 12, wherein the bladder outflow obstruction comprises benign prostatic hyperplasia (BPH).

14. The method of claim 7, wherein said therapeutically effective amount increases the residual volume by less than 15%.

15. The method of claim 8, wherein said therapeutically effective amount is sufficient to reduce the frequency by at least 20%.

16. The method of claim 7, wherein said netupitant is administered orally, in the form of a tablet, capsule or liquid, and said therapeutically effective amount comprises from 0.05 mg to 10 mg of netupitant, or its pharmaceutically acceptable salt or prodrug, per kg of the human patient.

17. A method of treating a human patient in need of treatment for bladder dysfunction, wherein said bladder dysfunction is selected from urgency, frequency, pollakiuria, nocturia, low deferment time, suboptimal volume threshold, and neurogenic bladder, or a combination thereof, comprising:
administering to said patient a therapeutically effective amount of netupitant to treat said dysfunction, or a pharmaceutically acceptable salt or prodrug thereof.

18. The method of claim 17, wherein said bladder dysfunction is selected from urgency, frequency, pollakiuria, nocturia, low deferment time, suboptimal volume threshold, and neurogenic bladder, or a combination thereof, wherein said amount is effective to treat said bladder dysfunction without significantly increasing residual volume.

19. The method of claim 17, wherein said netupitant is administered orally, in the form of a tablet, capsule or liquid, and said therapeutically effective amount comprises from 0.05 mg to 10 mg of netupitant, or its pharmaceutically acceptable salt or prodrug, per kg of the human patient.
